(19) United States
(12) Patent Application Publication
(10) Pub. No.: US 2025/0191754 A1
(43) Pub. Date: Jun. 12, 2025

(54) ARTIFICIAL INTELLIGENCE SYSTEM COMPATIBLE WITH VARIOUS TYPES OF INPUT DATA AND ARTIFICIAL INTELLIGEINCE MODELS WITHOUT CONFIGURING NEW PIPELINES

(71) Applicant: Pusan National University Industry-University Cooperation Foundation, Busan (KR)

(72) Inventors: Giltae Song, Yangsan-si (KR); Minwook Kim, Yangsan-si (KR); Jeonghoon Choi, Yangsan-si (KR); Juseong Kim, Yangsan-si (KR)

(21) Appl. No.: 18/823,719

(22) Filed: Sep. 4, 2024

(30) Foreign Application Priority Data
Dec. 12, 2023 (KR) 10-2023-0180016

(51) Int. Cl. G16H 50/20

(52) U.S. Cl. CPC G16H 50/20

(57) ABSTRACT

Provided is an improved artificial intelligence system compatible with various input data and artificial intelligence models without configuring new pipelines. This AI system improves efficiency by converting input data in a standardized format, and encapsulating common functions of artificial models, such that there is no need to create a new pipeline and thus efficiency of the system has been improved.

### CROSS-REFERENCE TO RELATED APPLICATIONS

This instant application claims priority to Korean Patent Application No. 10-2023-0180016, filed on Dec. 12, 2023, the disclosure of which is incorporated herein by reference in its entirety.

### BACKGROUND OF THIS INVENTION

This invention is related to an artificial intelligence system and prediction methods thereof. Specifically, this invention is related to an artificial intelligence system which can make use of various data and artificial intelligence models and prediction methods thereof.

Personal Health Record (PHR) data, which is a record of an individual's health, may contain various information. Using such PHR data for derivation of medical judgment, such as, a mortality rate of a present patient, is expanding from the realm of doctors to that of artificial intelligence. Usually, a medical diagnostic artificial intelligence draws to a conclusion by developing an artificial intelligence model for a specific type of data. That is, an independent pipeline is built according to the data, and a new pipeline is built when the artificial intelligence model is updated.

According to this conventional method, any possible overlapping data for each pipeline is discarded without being used again, and a completely new pipeline is required to be built, which leads to huge inefficiencies. Therefore, a lot of research has been conducted continuously to address these issues.

Additionally, myocardial infraction patients/heart failure patients have been diagnosed or prescribed uniformly in the same manner according to conventional cardiology clinical protocols regardless of individual characteristics of the patient. Such way of diagnosis or prescription is ineffective to some patients, and thus, a demand for a customized diagnosis or prescription to heart failure/myocardial infraction patients has been raised.

### SUMMARY OF THE INVENTION

One of the objectives of this invention is to provide an artificial intelligence system that does not need to be modified even if data and artificial intelligence models change.

Further, another objective of this invention is to provide a prediction method of an artificial intelligence system that does not need modification even if data and artificial intelligence models change.

Further, another objective of this invention is to provide a computer program stored in a recording medium that executes a prediction method of an artificial intelligence system that does not require modification even if data and artificial intelligence models change.

Further, another objective of this present invention is to provide technical assistance by facilitating a customized diagnosis or prescription for individual myocardial infraction/heart failure patients with an artificial intelligence system. With the customized diagnosis or prescription, an accurate diagnosis or prescription may be achieved and proper treatment to myocardial infraction/heart failure patients may be also available.

The objectives of this invention are not limited to the above mentioned, and other objectives and advantages of the present invention that are not mentioned herein can be understood by the examples of the present invention. Besides, the objectives and advantages of this present invention can be realized by the means and combinations thereof indicated in the present claims.

To address the above-mentioned technical issues, an artificial intelligence system according to some embodiments of the present invention for achieving the objectives is provided, which comprises a data software module for receiving the patient's PHR data and performing preprocessing of the PHR data, and generating the final data in a standardized format understandable or readable by the artificial intelligence system; a model software module for receiving the final data and an instruction and deriving a pre-prediction of the patient based on at least one artificial intelligence model selected according to the instruction; and Reliable and Interpretable Artificial Intelligence System (RIAS) operating independently from the data software module and the model software module, receiving the pre-prediction and the final data, and converting the pre-prediction into the final prediction.

Further, the patient may include a myocardial infraction patient.

Further, the final prediction may include the patient's mortality rate after a certain preset period of time.

Further, the final prediction may include the proportion of features (factors) positively contributing to the mortality rate and the proportion of factors negatively contributing to the mortality rate.

Further, the PHR data may include a mortality rate after an acute myocardial infraction of a patient and/or reverse remodeling data after an acute myocardial infraction of a patient.

The PHR data may further include data obtained after changing and/or manipulating the mortality rate and/or the reverse remodeling data.

Further, the data software module may comprise a preprocessing software module which receives an instruction including preset dataframe and preprocesses the PHR data to generate preprocessed data; and an imputation software module which processes missing values of the preprocessed data and generates final data.

Further, the imputation software module may receive the instruction and process the missing value according to the dataframe.

Further, the imputation software module may process the missing value through at least one of mean imputation, median imputation, mode imputation, regression imputation, K-nearest neighbor imputation, and multiple imputation.

Further, the instruction may include information with respect to the selected artificial intelligence model.

Further, if the at least one of artificial intelligence models has a common function, it can be classified into a single type, and the model software module can encapsulate the common function in accordance with the type.

Further, the type may include at least one of Gradient Boosted Decision Trees (GBDT), pytorch tabula and Saint.

Further, the RIAS may include a training software module training the artificial intelligence model, and tuning and evaluating a hyperparameter of the artificial intelligence model; a calibrating software module deriving a final prediction by correcting the pre-prediction; a local explanation (an instance-level factor contribution) software module generating local explanations on contribution, including each contributing factor and its proportion contributing to the final data; and a counterfactual explanation (counterfactual inference) software module generating counterfactual explanations or drawing an inference or an insight as to what change should be made to the factor(s) for obtaining the final prediction as desired by the user.

Further, the pre-prediction has a predicted value between 0 and 1, and the calibrating software module may derive the final prediction by adjusting the predicted value such that the pre-prediction imitates the probability of occurrence of an actual event. Imitation of the probability of the occurrence of an actual event may be performed by minimizing a value of Expected Calibration Error.

The artificial intelligence system providing a solution to the problem in accordance with the embodiments of this invention comprises a memory; a processor performing calculations in conjunction with the memory, and the processor generates final data by preprocessing PHR data of a patient, derives a pre-prediction by inputting the final data to a selected artificial intelligence model, and derives a final prediction by correcting the pre-prediction.

A prediction method of an artificial intelligence system providing a solution to other problems according to the embodiments of this present application, using the artificial intelligence system, comprises: a step of converting PHR data of a patient into final data by preprocessing the PHR data, a step of converting the final data based on a selected artificial intelligence model into a pre-prediction; and a step of deriving a final prediction by correcting the pre-prediction.

Further, the step of generating the final data may include a step of generating preprocessed data by converting the PHR data into a preset dataframe and generating the final data by processing the missing value of the preprocessed data, and may further include a step of receiving an instruction by the artificial intelligence system, wherein the instruction may comprise the preset dataframe.

Furthermore, the step of deriving the pre-prediction may include a step of selecting an artificial intelligence model according to the instruction, and deriving the pre-prediction based on the selected artificial intelligence model.

The artificial intelligence system and its prediction method based on the present invention do not require modification of a pipeline even if the input data changes. Additionally, even if the artificial intelligence model is changed, no modification of a pipeline is required, and thus, an effective setting up of a pipeline system may be achieved. Therefore, various types of input data and artificial intelligence models may be compatible with the artificial intelligence system according to an embodiment of this present invention. Further, based on this invention, factors contributing to the prediction may be pointed out, which makes it easier to understand and explain the prediction of the patient's health condition. Together with the above stated, specific advantageous effects of this present invention are described below with specific details for implementing this invention.

### BRIEF DESCRIPTION OF THE DRAWINGS

FIG. 1 is a block diagram explaining an artificial intelligence system according to some embodiments of the present invention.

FIG. 2 is a block diagram for explaining PHR data in FIG. 1.

FIG. 3 is a block diagram for explaining an instruction in FIG. 1.

FIG. 4 is an exemplary diagram for showing a prediction in which the beta-blocker in FIG. 1 is not administered.

FIG. 5 is an exemplary diagram for showing a prediction in which the beta-blocker in FIG. 1 is administered.

FIG. 6 is a block diagram which explains a structure of an artificial intelligence system in

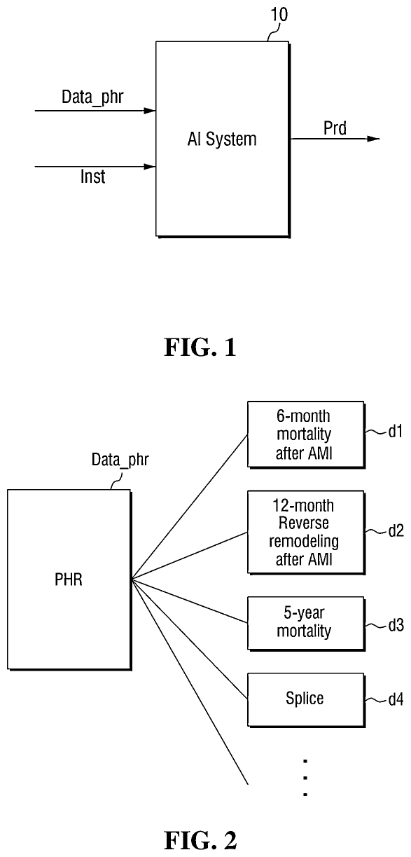

FIG. 1.

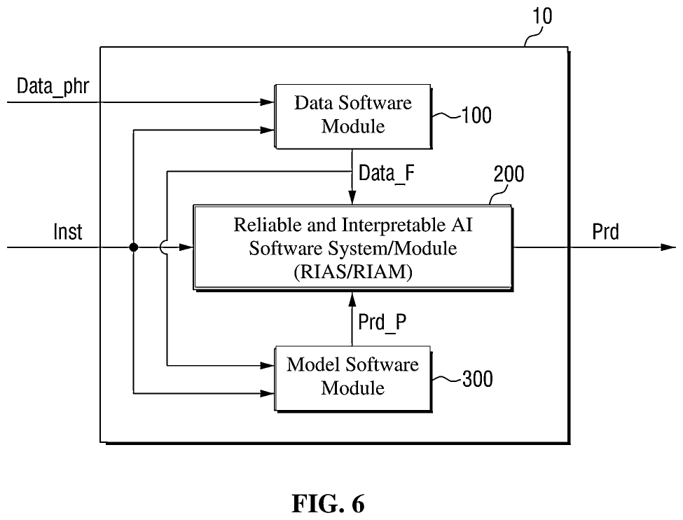

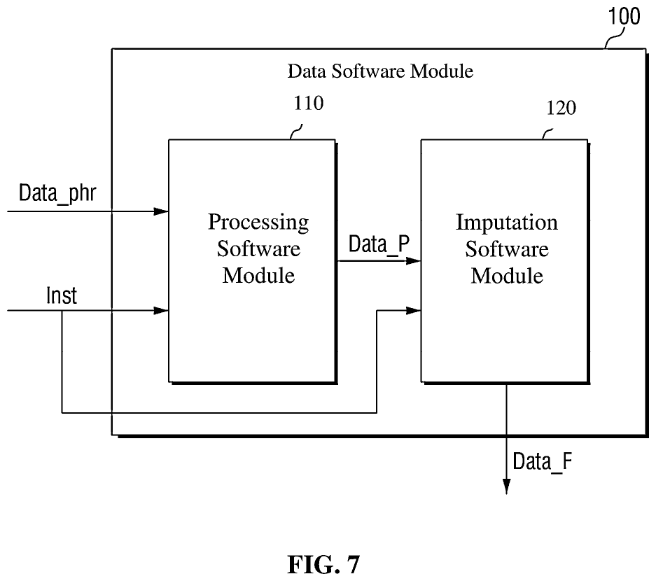

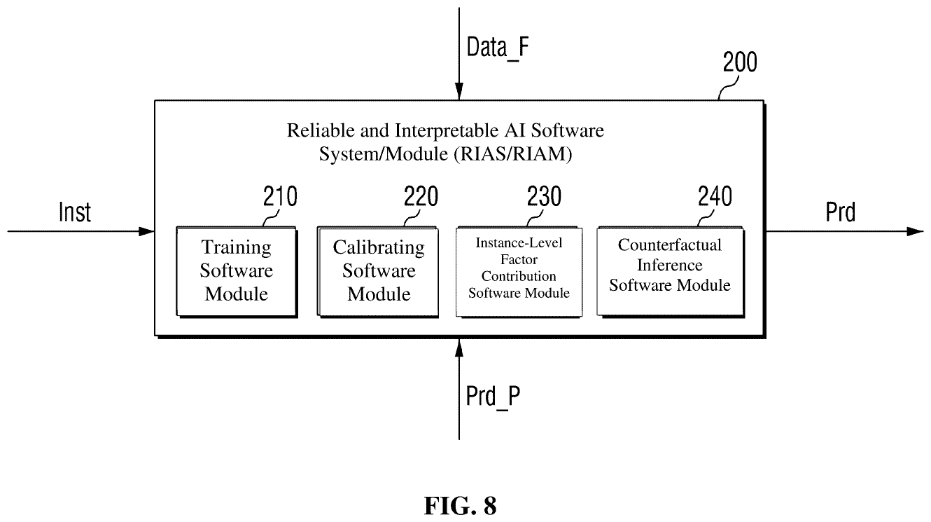

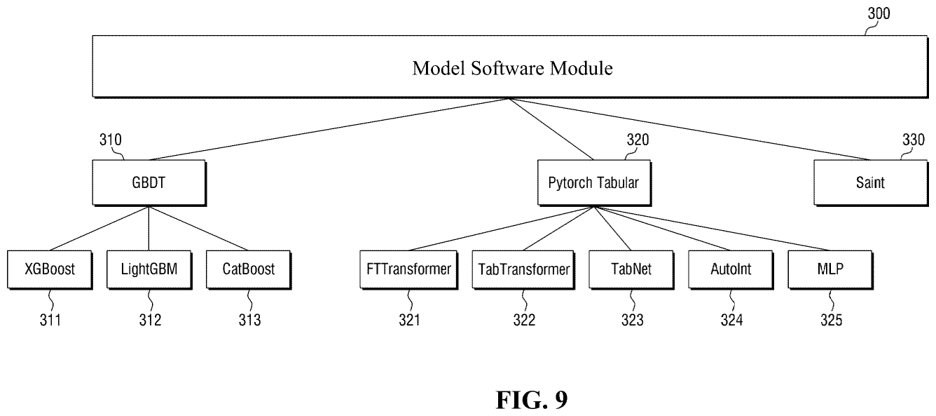

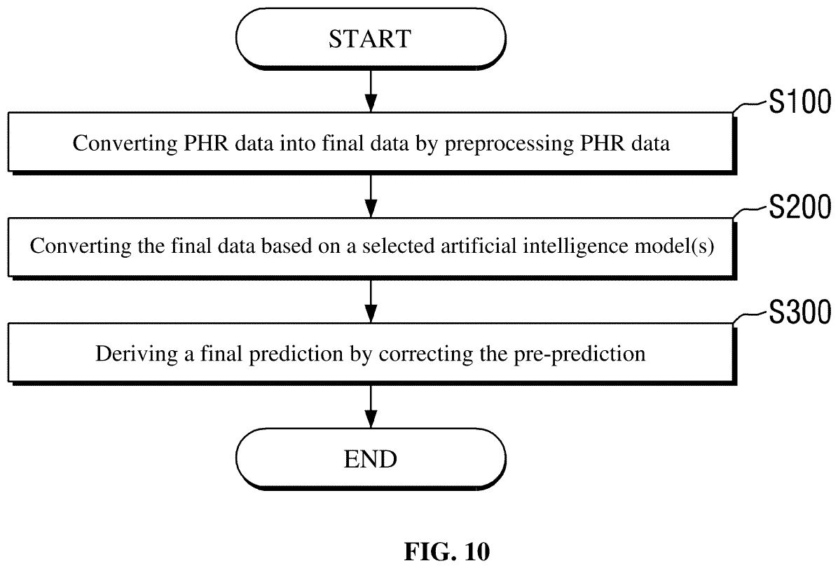

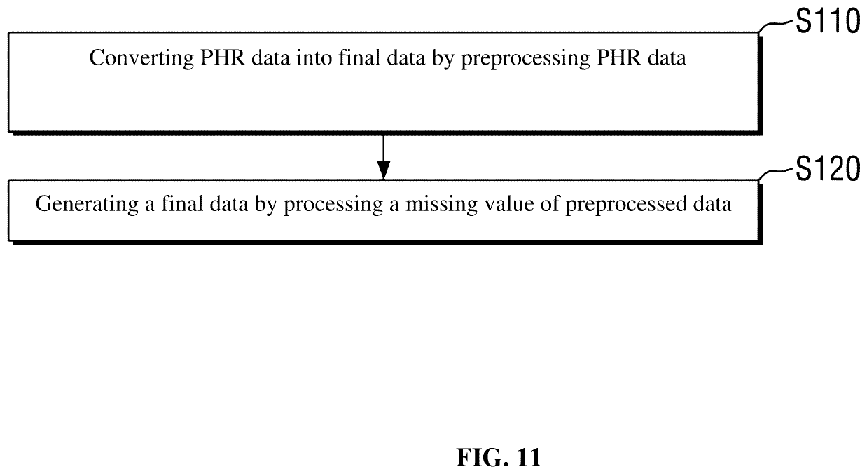

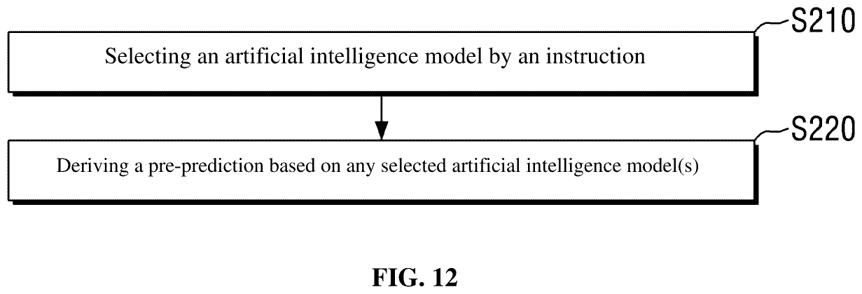

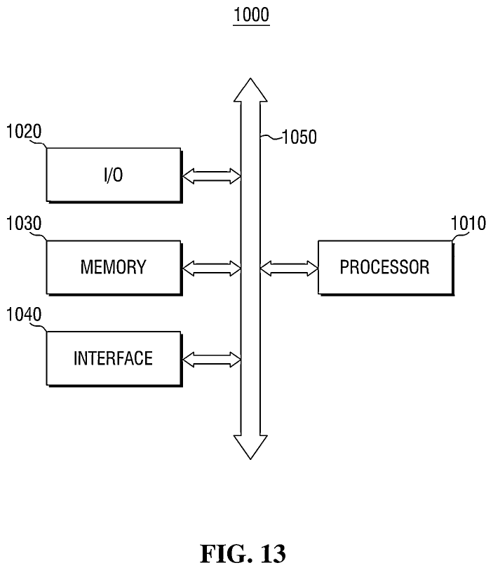

FIG. 7 is a block diagram for explaining a structure of a data software module in FIG. 6.

FIG. 8 is a block diagram for explaining a structure of RIAS in FIG. 6.

FIG. 9 is a block diagram for explaining a model software module in FIG. 6.

FIG. 10 is a flowchart for explaining a prediction method of an artificial intelligence system in accordance with the embodiments of this present invention.

FIG. 11 is a flowchart explaining a step of generating final data in FIG. 10 in detail.

FIG. 12 is a flowchart explaining a step of deriving a pre-prediction in FIG. 10 in detail.

FIG. 13 is a diagram explaining hardware configuration of an artificial intelligence system in accordance with the embodiments of this present invention.

### DETAILED DESCRIPTION

Terms or words used in this specification and the claims shall not be construed as limited to their general dictionary meaning. The inventor may define a term, a word, or a concept in order to best explain his or her invention, and according to the principle, the specification and claims should be interpreted with the meaning and concept in consistent with the technical idea of the present invention. In addition, the embodiments described in this specification and the configurations illustrated in the drawings are only an example of the present invention and do not completely encompass the technical idea of the present invention, and therefore, it should be understood that any equivalents, variations, or any other examples of applications may replace the described embodiments at the filing of this present application.

The terms “first”, “second”, “A” and “B” and the like may be used to describe various elements, but do not limit the elements. Such terms are only used to classify one element from another. For example, “a first component” may be named “a second component”, without departing from the scope of the present invention, and similarly, “a second component” may be named “a first component”. The term ‘and/or’ includes a combination of a plurality of related terms previously stated or any of a plurality of related terms previously stated.

The terms in this specification and claims are merely used to describe a specific embodiment of this present invention, and the use of the terms are not intended to limit the present invention. An expression with a singular term includes an expression of plural terms unless otherwise clearly indicated by the context. The terms “include”, “have” and the like in the present application must be understood not to exclude the possibility of existence or addition of features, numbers, steps, operations, components, parts, or combination thereof described in the specification.

Unless otherwise defined, all the terms, including technical or scientific terms and the like, used in this present application have the same meaning as generally understood by an ordinary person skilled in the pertinent art.

The terms that are commonly defined in dictionaries should be interpreted in consistent with the meanings they have in the context of the related art, and unless otherwise clearly defined in this application, the terms should not be interpreted in a manner idealistic or excessively formal.

Moreover, each configuration, course, process, method and the like included in each embodiment of the present invention may be shared within the scope of not being technically contradictory to each other.

Hereinafter, in reference to FIGS. 1 to 9, an artificial intelligence system in accordance with some embodiments of the present invention is described.

FIG. 1 is a block diagram for explaining the artificial intelligence system in accordance with the embodiments of the present invention.

Referring to FIG. 1, an artificial intelligence system (10) according to embodiments of this present invention may receive PHR data (DATA_PHR) and an instruction (Inst). The instruction may be in a form of a command, software, or a code. The artificial intelligence system (10) may internally generate a final prediction (Prd) through the PHR data (DATA_PHR) and the instruction (Inst). The artificial intelligence system (10) may transmit the generated final prediction (Prd) to an outside.

FIG. 2 is a block diagram illustrated to explain the PHR data in FIG. 1.

Referring to FIG. 2, the PHR data (DATA_PHR) may be data with respect to various health records of a patient. For example, the PHR data (DATA_PHR) may be individual health record data, however, it is not limited thereto. More specifically, the PHR data (DATA_PHR) may include at least one of first data (d1) on a mortality rate during 6 months from the onset of acute myocardial infraction (AMI), second data (d2) on reverse remodeling during 12 months from the onset of the acute myocardial infraction, third data (d3) on mortality rate during 5 years from the onset of the acute myocardial infraction and fourth data (d4) obtained by changing or manipulating the above stated data.

Here, the reverse remodeling is a process through which the heart recovers from damage caused by the myocardial infraction, and may encompass a process by which the size, shape, and function of the heart gets recuperated or returns to its normal condition. In other words, the process of the reverse remodeling includes any medical strategies designed for minimizing damages to heart tissues and optimizing heart function such that the patient's life quality is improved.

The periods mentioned above for the first data (d1), the second data (d2) and the third data (d3) are all used as examples, and any other period may be used depending on need and purpose.

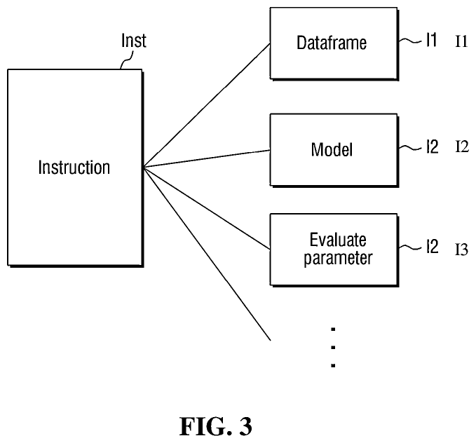

FIG. 3 is a block diagram used for explaining the instruction in FIG. 1.

Referring to FIGS. 1 to 3, the instruction (Inst) may include various information necessary for operating the artificial intelligence system (10) according to embodiments of this invention.

For example, the instruction (Inst) may include at least one of first information (11) about dataframe, second information (12) about artificial intelligence models, and third information (13) about evaluation parameters for evaluating artificial intelligence models. And the evaluation parameters include accuracy, F1-Score, precision, recall, and the like.

At this time, the first information (11) may be information on how to preprocess a dataframe of the PHR data (DATA_PHR). In other words, the PHR data (DATA_PHR) may exist in various formats, and thus, if it is not converted into a single dataframe, inefficiency to construct a new pipeline system can take place. The artificial intelligence system (10) according to one of the embodiments converts various PHR data (DATA_PHR) into a preset dataframe included in the instruction (Inst) and transmits it to the artificial intelligence model, and thus, efficiency can be maximized as no need to configure a new pipeline system.

The second information (12) may be information designating the artificial intelligence model. In other words, information about the intelligence model to be used to derive the final prediction (Prd) may be included in the second information (12). When it is required to introduce a new or updated artificial intelligence model, conventional prediction systems may experience inefficiencies as they require a new pipeline system. The artificial intelligence system (10) according to the embodiment of this present invention can improve efficiency by selecting an artificial intelligence model as a preset according to the instruction (Inst) and by using functions that overlap with other artificial intelligence models.

The third information (13) may be required when presetting parameters necessary for evaluating artificial intelligence models. In such a case, the third information (13) may be preset so as to allow any selected artificial intelligence model among various artificial intelligence models to be applied.

The instruction may be in a form of a command, software, or a code.

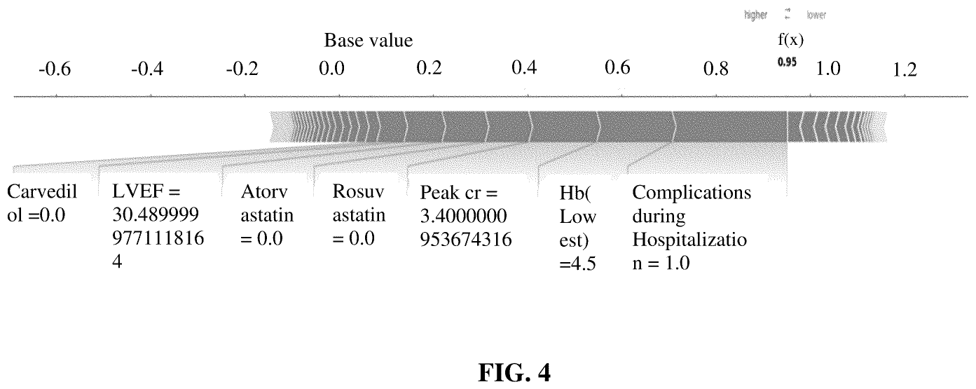

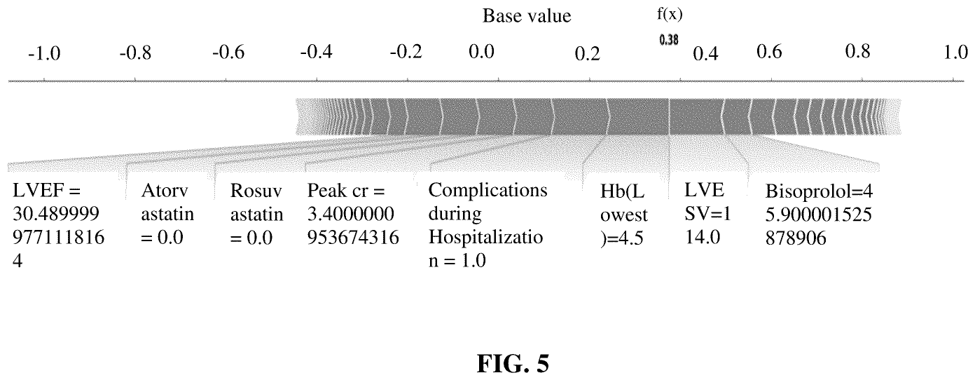

FIG. 4 is an exemplary diagram illustrating the final prediction when the beta-blocker in FIG. 1 is not administered, and FIG. 5 is an exemplary diagram illustrating the final prediction when the beta-blocker in FIG. 1 is administered.

Referring to FIGS. 1 and 4-5, for example, the final prediction (Prd) may represent a probabilistic value of the patient's mortality rate after 12 months from the onset of the myocardial infraction. FIG. 4 shows an example of the final prediction (Prd) that the mortality of the patient's mortality rate is 95% if the beta-blocker was not administered after the onset of the myocardial infraction.

The left-side (red) bar in FIG. 4 may denote the patient's conditions or factors contributing to the patient's death, and the right-side (blue) bar in FIG. 4 may denote the patient's conditions or factors contributing to the patient's survival. The final prediction (Prd) of the artificial intelligence system (10) according to this embodiment may provide proportions of contribution to the current final prediction (Prd) value, which facilitates analysis and understanding. The factors contributing to the patient's death or survival may include development of complications during hospitalization, lowest hemoglobin level, highest creatinine level, and the like.

Referring to FIG. 5, it can be seen that the mortality rate gets reduced to 38% when the beta-blocker is administered. That is, a medical professional in charge of treating the patient can get assistance with diagnosis and prescription by comparing the final prediction (Prd) in FIG. 4 with the final prediction (Prd) in FIG. 5.

FIG. 6 is a block diagram explaining the structure of the artificial intelligence system in FIG. 1.

Referring to FIG. 6, the artificial intelligence system (10) may comprise a data software module (100), a Reliable and Interpretable Artificial Intelligence System (RIAS) (200) and a model software module (300).

The data software module (100) may receive the PHR data (DATA_PHR) and the instruction (Inst) and perform preprocessing. The data software module (100) may preprocess the PHR data (DATA_PHR) according to the instruction (Inst), and then, generate a final data (Data_F). The data software module (100) may transmit the final data (Data_F) to the RIAS (200) and the model software module (300).

The model software module (300) may learn artificial intelligence models and perform a prediction after receiving the instruction (Inst) and the final data (Data_F). The model software module (300) may generate a pre-prediction (Prd_P) through the artificial intelligence models. The model software module (300) may generate the pre-prediction as follows. When the user requests to generate a prediction result for a specific data sample, a pre-prediction is generated for the given data sample using a pre-trained model. The pre-trained model refers to a model which has learned data provided by the user. The process of creating the pre-trained model by learning the machine learning model is as follows: (1) preprocessing is performed so that the dataset provided by the user can be processed by the machine learning; (2) finding out the machine learning model that shows the best performance for the user's desired task based on the preprocessed dataset; and (3) training the found machine learning model to create the final machine learning model.

Pre-prediction is created along with the most likely class for a given prediction task and the degree of confidence in the prediction results of the artificial intelligence model. For instance, if there are two classes, patient's death and survival, and the artificial intelligence model determines that there is a 70% chance of death based on the given data, it generates a pre-prediction stating that there is a 70% likelihood of death.

An artificial intelligence model typically calculates the likelihood which class a given data sample belongs to. Therefore, the most likely class can be intuitively determined based on calculation by the artificial intelligence model. Likewise, the degree of confidence in the prediction result may be obtained by interpreting the probability resulting from the calculation by the artificial intelligence model as the degree of confidence.

The model software module (300) may transmit the pre-prediction (Prd_P) to the RIAS (200). In such a case, the value of the pre-prediction may be between 0 and 1.

The RIAS may include Reliable and Interpretable Artificial Intelligence Modules (RIAM). The RIAS/RIAM (200) may receive the instruction (Inst) and the final data (Data_F). The RIAS (200) may also receive the pre-prediction (Prd_P) from the model software module (300). The RIAS (200) may derive a final prediction (Prd) from the pre-prediction (Prd_P) after using the final data (Data_F) and the instruction (Inst). The RIAS (200) may evaluate and tune the pre-prediction (Prd_P).

Here, the data software module (100), the model software module (300) and the RIAS (200) may operate regardless of the types of the data and the artificial intelligence models. That is, artificial intelligence systems, in general, generate predictions with a predetermined artificial intelligence model in accordance with the type of data, and if the data or the artificial intelligence model is changed, a new pipeline system must be implemented, instead of using a conventional pipeline system.

In contrast, according to the artificial intelligence system according to this embodiment, the data software module (100) provides the data after converting them into the same dataframe in the standardization format, and the model software module (300) performs encapsulation so that it may utilize common functions among various artificial intelligence models, such that the RIAS (200) may derive the prediction (Prd) independently of data and artificial intelligence models without configuration of a new pipeline system. Encapsulation is a practice of bundling data and methods within a single unit, like a class, and controlling their access, and encapsulation secures data and functions within a class, preventing unauthorized access and modification. In this invention, the term encapsulation may refer to allowing an artificial intelligence system to operate an artificial intelligence model through an abstract interface so that the artificial intelligence system can be used with the same logic even if the artificial intelligence model is changed. An abstract interface is an abstraction representing more than one interface and may describe common aspects of systems of that type but may omit the aspects that distinguish from each other. With the encapsulation process, the artificial intelligence system may derive the prediction independently of artificial intelligence models, and may also prevent unauthorized access and modification enhancing an accuracy of the artificial intelligence system.

The RIAS generates an optimized artificial intelligence model for a given dataset and task (for example, the task of predicting mortality for patients with myocardial infraction). Machine learning techniques used herein include Random Forest, Light Gradient Boosting Machine, FT-Transformer, and the like. And a confidence calibration is performed by the RIAS so that a predicted value of the trained machine learning imitates an actual probability. Afterwards, the RIAS may generate a predicted value that can be interpreted as an actual probability, determine how much certain factors of the data contribute to the prediction, and additionally obtain an insight such as a counterfactual inference, about what change in the factor(s) of the data should be made to change the prediction result of an artificial intelligence system.

FIG. 7 is a block diagram explaining the structure of the data software module in FIG. 6.

Referring to FIG. 7, the data software module (100) may include a preprocessing software module (110) and an imputation software module (120).

The preprocessing software module (110) may receive the PHR data (DATA_PHR) and the instruction (Inst). At this time, the instruction (Inst) may include information about the dataframe. The preprocessing software module (110) may preprocess the PHR data (DATA_PHR) through the information included in the instruction (Inst). Therefore, the preprocessing software module (110) may generate the preprocessed data (Data_P). Generally, raw data is in a format difficult for computers to process, and therefore, raw data needs preprocessing by being converted into a computer-friendly format in accordance with an embodiment of the present disclosure. For example, blood pressure data should be treated as data of figures (numbers/numeric data), however, it may be treated as a string data. Therefore, in such a case, any possible string data should be preprocessed so that they may be treated as numeric data. In addition, since raw data is generally collected by humans, incorrect values may be collected during the process. Thus, in the preprocessing step, these incorrect values may be eliminated or replaced by normal values, which helps the computer easily understand or read the raw data.

More specifically, data, such as key data, may include a unit, such as, feet or centimeter, as well as numbers in the dataframe. In such a case, since the key data cannot be treated as a numeric data as is, it should be converted into a numeric data by removing the unit which may cause a problem in the data. Further, the PHR data may include typographical errors in the names of the administered drugs, treatment methods, or the same drug may be named differently depending on a person who puts down. In such a case, these data may be preprocessed by correcting the typographical errors and unifying the different names. In addition, due to an error occurred during the course of data collection, there may be abnormal numeric data that cannot be held by humans. Instead of making use of such abnormal data, they may be preprocessed by not processing or treating them in the first place.

Lastly, some particular factors of the PHR data may be omitted because such factors are not properly collected. If the omitted rate is too high, it may not yield a meaningful value, and therefore, the preprocessing process may be necessary to remove these factors having high omitted rates.

In other words, the data software module (100) performs standardization of input data so that they are easily understood or read by artificial intelligence models.

The imputation software module (120) may receive the preprocessed data (Data_P) from the preprocessing software module (110). Moreover, the imputation software module (120) may determine a manner of processing a missing value included in the instruction (Inst) after receiving the instruction (Inst).

For example, the imputation software module (120) may process the missing value through at least one of methods of mean imputation, median imputation, mode imputation, regression imputation, K-nearest neighbor imputation, and multiple imputation. In such a case, the method(s) selected by the imputation software module (120) may be preset method(s), or designated method(s) by the instruction (Inst). The imputation software module (120) may generate the final data (Data_F) after eliminating the missing value of the preprocessed data (Data_P).

FIG. 8 is a block diagram explaining the structure of the RIAS in FIG. 6.

Referring to FIG. 8, the RIAS/RIAM (200) may comprise at least one of a training software module (210), a calibrating software module (220), an instance-level factor contribution software module (230), and a counterfactual inference software module (240).

The training software module (210) may inherit, from the model software module (300), the model of the pre-prediction (Prd_P) received by the RIAS (200), and then, tune, learn and evaluate hyperparameters of the model. The training software module (210) may operate as is even if the artificial intelligence model selected by the model software module (300) changes.

The calibrating software module (220) may convert the pre-prediction (Prd_P) into the final prediction (Prd). The calibrating software module (220) may process the pre-prediction (Prd_P) to imitate the probability of occurrence of an actual event and convert it into the prediction (Prd), such that a user may accept it as the expected probability of occurrence of the actual event. Specifically, the degree of confidence an artificial intelligence model has in its predictions may be measured by a value of Expected Calibration Error (ECE). The calibrating software module (220) calibrates the pre-prediction in a way the ECE value is minimized, and thus, the imitation of the probability of occurrence of an actual event may be achieved.

The ECE may be measured by the process of (1) generating the prediction results of the artificial intelligence model for the given dataset; (2) grouping the level of confidence in the prediction result generated by the artificial intelligence model into specific intervals (for instance, it can be grouped as 100%-90%, 90%-80%, . . . with 10% intervals for each group); and (3) finding the difference between the average accuracy and average confidence level for each section and calculate the average of the difference for each section to obtain the final ECE. For example, if the average accuracy for the prediction result which falls within the range of 100%-90% is 95%, it can be said that an ideal result is generated. In such a method, the calibrating module may tune the ECE to be minimized by adjusting the degree confidence the artificial intelligence model has in the prediction result through the selected calibration method.

And the calibrating method may include temperature scaling and histogram binning.

Temperature scaling allows the artificial intelligence model to adjust the degree of confidence in the prediction result by dividing the output value immediately before generating it by a specific constant (tau), and can be tuned by adjusting the value of tau.

Histogram binning is a method of grouping the degree of confidence in an artificial intelligence model's prediction into a specific interval and changing all the degrees of confidence within that interval to a specific representative value. The degree of confidence may be adjusted and tuning can be done by adjusting the representative value for each section.

The instance-level factor contribution software module (230) may inform how much each factor of the final data (Data_F) used in the prediction (Prd) has contributed to the prediction (Prd). That is, the instance-level factor contribution software module (230) may generate the contribution factors and their proportions as shown in FIGS. 4 and 5.

And a counterfactual inference software module (240) may provide an insight or an inference as to what the user should do to obtain the desired result based on the pre-prediction (Prd_P). Accordingly, the prediction (Prd) may include the insight. For example, an insight that confirms mortality rates with and without administering the beta-blocker in FIGS. 4 and 5 and provides a suggestion of administering the beta-blocker or prescribing medication may be provided by the counterfactual inference software module (240). For finding out what change or modification of factor(s) may change the prediction, a machine learning process may be used.

The RIAS may be a framework which allows a user to trust and interpret machine learning when they apply machine learning to a specific task. All the functions performed by the RIAS may be related to machine learning. Basically, when generating a prediction result through RIAS, machine learning is performed to learn a specific dataset in advance and generate the most likely prediction result based on what the RIAS has been learned.

Machine learning may be used when creating a SHAP (SHapley Additive explanations) value to explain the prediction result, when generating a counterfactual explanation or counterfactual inference as to which elements of a given data must be changed and how the change should be made to obtain a different predicted result of the artificial intelligence model, and when calibrating to generate the prediction result.

FIG. 9 is a block diagram used for explanation of the model software module in FIG. 6.

Referring to FIG. 9, the model software module (300) may select one among various artificial intelligence models and perform learning and inference. Each model may be classified into the same type as models having the common functions. Specifically, the artificial intelligence model may include a first type (310), a second type (320), and a third type (330). In FIG. 9, the number of types is described as three. However, the embodiment of the present invention is not limited thereto.

For example, the first type (310), the second type (320), and the third type (330) may be Gradient Boosted Decision Trees, Pytorch tabula and Saint, respectively, which is, however, merely an example and the embodiment of the present invention is not limited thereto.

Accordingly, a first model (311), a second model (312), and a third model (313) that belong to the first type (310) may be XGBoost, LightGBM and CatBoost, respectively. However, the embodiment of the present invention is not limited thereto. A fourth model (321), a fifth model (322), a sixth model (323), a seventh model (324), and an eighth model (325) may be FTTransformer, TabTransformer, TabNet, AutoInt, and MLP, respectively. However, the embodiment of the present invention is not limited thereto.

In this way, the model software module (300) may perform encapsulation to specify a common object for the same type of artificial intelligence models with the same function such that the pipeline is shared as much as possible.

This embodiment operates independently of data or artificial intelligence models, and therefore, the same flow may be used no matter what data or models are used. Accordingly, the prediction may be derived far more efficiently unlike a conventional artificial intelligence system.

Hereinafter, referring to FIGS. 6 to 8 and FIGS. 10 to 12, a prediction method of the artificial intelligence system according to some embodiments of this present invention is explained. Parts overlapping with the above-described embodiments are simplified or skipped.

The prediction method of the artificial intelligence system according to the embodiments of this present invention may be stored to a recording medium and executed as a computer program. In such a case, the operation may be performed by organically combining with the artificial intelligence system. However, the embodiment of the present invention is not limited thereto.

FIG. 10 is a flow diagram for explaining the prediction method of the artificial intelligence system according to some embodiments of this present invention, and FIG. 11 is a flow diagram for explaining, in detail, a step of generating the final data in FIG. 10. FIG. 12 is a flow diagram for explaining, in detail, a step of deriving the pre-prediction in FIG. 10.

Referring to FIG. 10, the final data may be obtained by converting the PHR data in a standardized format readable by the artificial intelligence system (S100).

In detail, referring to FIG. 11, the preprocessed data is generated by converting the PHR data into the preset dataframe (S110).

Specifically, referring to FIG. 7, the preprocessing software module (110) may receive the PHR data (DATA_PHR) and the instruction (Inst). At this time, the instruction (Inst) may include information about the dataframe. The preprocessing software module (110) may preprocess the PHR data (DATA_PHR) through the information with respect to the dataframe included in the instruction (Inst). Accordingly, the preprocessing software module (110) may convert the PHR data (DATA_PHR) into the preprocessed data (Data_P).

Again, referring to FIG. 11, the final data may be obtained by processing the missing value of the processed data (S120).

Specifically, referring to FIG. 7, the imputation software module (120) may receive the preprocessed data (Data_P) from the preprocessing software module (110). Additionally, the imputation software module (120) may receive the instruction (Inst) and determine the method of processing the missing value included in the instruction (Inst). The imputation software module (120) may convert the preprocessed data and into the final data (Data_F) by eliminating the missing value of the preprocessed data (Data_P).

Again, referring to FIG. 10, the pre-prediction may be derived by converting the final data based on a selected artificial intelligence model(s) (S200).

Referring to FIG. 12 in detail, the artificial intelligence model(s) is selected by the instruction (S210), and the pre-prediction is derived through the selected artificial intelligence model(s) (S220).

Specifically, referring to FIG. 6, the model software module (300) may learn the artificial intelligence model and perform prediction after receiving the instruction (Inst) and the final data (Data_F). In learning the selected artificial intelligence model, the model software module (300) may make use of the encapsulation specifying common functions among the selected artificial intelligence models. The model software module (300) may generate the pre-prediction (Prd_P) through the artificial intelligence model. The model software module (300) may transmit the pre-prediction (Prd_P) to the RIAS (200).

Again, referring to FIG. 10, the final prediction is derived by correcting the pre-prediction (S300).

Specifically, referring to FIG. 8, the calibrating software module (220) may convert the pre-prediction (Prd_P) into the final prediction (Prd). The calibrating software module (220) may process the pre-prediction (Prd_P) to imitate the probability of occurrence of an actual event and convert it into the final prediction (Prd) so that the user may accept it as an expected probability of occurrence of the actual event. Specifically, such process may be performed by adjusting the pre-prediction through minimizing the ECE value.

FIG. 13 is a diagram for explaining a configuration of hardware of the artificial intelligence system according to some embodiment of this present invention.

The artificial intelligence system according to the embodiments of this present invention may be implemented by an electronic device (1000). The electronic device (1000) may comprise a processor (1010), an input/output device (1020, I/O), a memory (1030), an interface (1040) and a bus (1050). The processor (1010), the input/output device (1020), the memory (1030) and/or the interface (1040) may be connected to each other by the bus (1050). The bus (1050) is a path through which data move.

Specifically, the processor (1010) may include at least one of a central processing unit (CPU), a micro processor unit (MPU), a graphic processing unit (GPU), a microprocessor, a digital signal processor, a microcontroller, an application processor (AP), etc. and any other logic elements capable of performing similar functions.

The processor (1010) and the memory (1030) in FIG. 1 may be configured as software on the electronic device (1000), or as hardware separately.

The input/output device (1020) may include at least one of a keypad, a keyboard, a touchscreen and a display device.

The memory (1030) may load data, programs and/or the like. At this time, the memory (1030) is a memory for enhancing the operability of the processor (1010), and may include high-speed dynamic random access memory (DRAM), a static random access memory (SRAM), and the like. The memory (1030) may include one or more of volatile memory devices, such as a double data rate static DRAM (DDR SDRAM), and a single data rate SDRAM (SDR SDRAM), and/or a non-volatile memory device, such as, an electrical erasable programmable read-only memory (EEPROM), and a flash memory.

The interface (1040) may transmit and receive data with a communication network. The interface (1040) may be wired or wireless. For instance, the interface (1040) may include an antenna, a wired or wireless transceiver, and the like.

A storage may store data, programs, and/or the lie. The storage may include at least one of a solid state drive (SSD), a hard drive, a flash drive, and the like. The storage in the present invention may store a computer program comprising an instruction which includes the above-mentioned preset dataframe and the preset artificial intelligence model(s).

The input/output device (1020) may encompass a personal digital assistant (PDA), a portable computer, a web tablet, a wireless phone, a mobile phone, a digital music player, a memory card, and any other electronic products that can transmit and/or receive in a wireless environment.

Further, a quantum circuit simulation device according to the embodiments of the present invention may be a device configured by connecting a plurality of electronic devices (1000) to each other through a network. However, the embodiment of the present invention is not limited thereto.

Additionally, the quantum circuit simulation device can be implemented by at least one of a workstation, a data center, an internet data center (IDC), a direct attached storage system (DAS), a storage area network system (SAN), a network attached storage system (NAS), and a redundant array of inexpensive disks, or redundant array of independent disks (RAID) system, however, the embodiment is not limited thereto.

Further, the quantum circuit simulation device can transmit data through the network. The network may include networks based on wired Internet technology, wireless Internet technology, and short-distance communication technology. For example, the wired Internet technology may include at least one of a Local area network (LAN) and a wide area network (WAN).

The description above is merely for an illustrative explanation of the technical idea of the present embodiments, and those skilled in the art may be able to make various changes and variations without departing from the essential characteristics of the present embodiment. Accordingly, it is noted that these embodiments are not limiting the technical idea of the present embodiments, but rather they are for explanation, and thus, the scope of the technical idea of the present embodiments is not limited thereto. The scope of protection based on these embodiments should be interpreted in accordance with the claims below, and all technical ideas within the equivalent scope should be interpreted as being included within the scope of rights of these embodiments.

1. An artificial intelligence system compatible with various types of input data and artificial intelligence models and including a pipeline, the system comprising:
at least one processor; and
at least one memory including computer program code,
wherein the computer program code includes a data software module, a model software module, and a reliable and interpretable artificial intelligence module (RIAM),
wherein the data software module, when executed by the at least one processor, is configured, with the at least one processor, to cause the system to receive an instruction including a preset dataframe and information selecting at least one artificial intelligence model among the artificial intelligence models, and to cause the system to receive data and to preprocess the data so as to convert the data, according to the preset dataframe, into final data in a standardized format readable by the artificial intelligence system;
wherein the model software module, when executed by the at least one processor, is configured, with the at least one processor, to cause the system to receive the final data and the instruction,
wherein, the model software module is configured to learn the selected at least one artificial intelligence model, and
wherein, when the selected at least one artificial intelligence model has one or more common functions, the at least one artificial model is classified as a type by the model software module, and the model software module encapsulates the one or more common functions in accordance with the type, such that the system is compatible with the various types of input data and the artificial intelligence models and removes inefficiency of configuring a new pipeline.

2. The system of claim 1, wherein the received and converted data is personal health record (PHR) data of a patient.

3. The system of claim 2, wherein the model software module is configured to derive a pre-prediction of health of the patient based on the PHR data and the selected at least one artificial intelligence model, and the RIAM is configured to receive the pre-prediction and the final data and to convert the pre-prediction into a final prediction.

4. The artificial intelligence system of claim 3,
wherein the RIAM includes at least one of:
a training software module configured to train the selected at least one artificial intelligence model, and tune and evaluate a hyperparameter of the selected at least one artificial intelligence model,
a calibrating software module configured to derive the final prediction by correcting the pre-prediction,
an instance-level factor contribution software module configured to present a contributing factor and a proportion of the contributing factor with respect to the final data, and
a counterfactual inference software module configured to generate a counterfactual inference as to what change in the factor may change the final prediction.

5. The artificial intelligence system of claim 4,
wherein the pre-prediction has a predicted value between 0 and 1, and
wherein the calibrating software module is configured to derive the final prediction by adjusting the predicted value through minimizing a value of Expected Calibration Error such that the pre-prediction imitates a probability of occurrence of an actual event.

6. The artificial intelligence system of claim 4,
wherein the counterfactual inference includes prescribing medication and administering a drug.

7. The artificial intelligence system of claim 6,
wherein the drug includes a beta-blocker.

8. The artificial intelligence system of claim 2,
wherein the data software module includes:
a preprocessing software module configured to receive the instruction, and to convert the PHR data into preprocessed data based on the preset dataframe, and
an imputation software module configured to convert the preprocessed data into the final data by processing a missing value of the preprocessed data.

9. The artificial intelligence system of claim 8,
wherein the imputation software module receives the instruction and processes the missing value according to the preset dataframe, and
wherein the imputation software module is configured to process the missing value based on at least one of mean imputation, median imputation, mode imputation, regression imputation, K-nearest neighbor imputation, and multiple imputation.

10. The artificial intelligence system of claim 1,
wherein the type includes at least one of gradient boosted decision trees (GBDT), pytorch tabula and Saint.

11. The artificial intelligence system of claim 2, wherein the patient includes a myocardial infraction patient.

12. The artificial intelligence system of claim 3,
wherein the final prediction includes a mortality rate of the patient after a preset period time.

13. The artificial intelligence system of claim 12, wherein the final prediction includes a factor positively contributing to the mortality rate and a factor negatively contributing to the mortality rate.

14. The artificial intelligence system of claim 2,
wherein PHR data include at least one of the mortality rate after an acute myocardial infraction and reverse remodeling data of the patient after the acute myocardial infraction.

15. The artificial intelligence system of claim 14,
wherein the PHR data further include at least one of data changed or manipulated from the mortality rate after the acute myocardial infraction and data changed or manipulated from the reverse remodeling data.

16. A method for improving efficiency of an artificial intelligence system compatible with various types of input data and artificial intelligence models and including a pipeline, the method comprising:
receiving an instruction including a preset dataframe and information selecting at least one artificial intelligence model;
selecting the at least one artificial intelligence model among the artificial intelligence models according to the instruction;
receiving data and converting the data into final data in a standardized format readable by the artificial intelligence system by preprocessing the data;
receiving the final data and the instruction and learning the selected at least one artificial intelligence model,
wherein, when the selected at least one artificial model has one or more common functions, the at least one artificial model is classified as a type by the model software module, and the model software module encapsulates the one or more common functions in accordance with the type, such that the system is compatible with the various types of input data and the artificial intelligence models and removes inefficiency of configuring a new pipeline.

17. The method of claim 16, wherein the received and converted data is personal health record (PHR) data of a patient.

18. The method of claim 17, further comprises:
deriving a pre-prediction of health of the patient based on the PHR data and the selected at least one artificial intelligence model; and
correcting and converting the pre-prediction into a final prediction.

19. The method of claim 16, further comprises:
processing of a missing value of the preprocessed data, according to the instruction, by one of mean imputation, median imputation, mode imputation, regression imputation, K-nearest neighbor imputation, and multiple imputation,
wherein the instruction further includes information as to the processing of the missing value.

20. The method of claim 16,
wherein the correcting of the pre-prediction is performed by adjusting the pre-prediction through minimizing a value of Expected Calibration Error such that the pre-prediction imitates a probability of occurrence of an actual event.

21. A computer program stored to a non-transitory computer-readable medium and configured to execute the method of claim 16.
